(19) United States
(12) Patent Application Publication
(10) Pub. No.: US 2025/0275974 A1
(43) Pub. Date: Sep. 4, 2025

(54) TREATMENT OF FRONTAL FIBROSING ALOPECIA

(71) Applicant: LEO PHARMA A/S, Ballerup (DK)

(72) Inventors: Adam RAFF, Needham, MA (US); Maryanne SENNA, Boston, MA (US); Anders Bacher NIELSEN, Ballerup (DK)

(21) Appl. No.: 18/857,877

(22) PCT Filed: Apr. 17, 2023
(86) PCT No.: PCT/EP2023/059929

§ 371 Date: Oct. 18, 2024

Related U.S. Application Data
(60) Provisional application No. 63/363,266, filed on Apr. 20, 2022.

(51) Int. Cl. A61K 31/519

(52) U.S. Cl. CPC A61K 31/519

(57) ABSTRACT

The present disclosure relates to the treatment of cicatricial alopecia, lichen planopilaris, and frontal fibrosing alopecia with 3-[(3S,4R)-3-methyl-6-(7H-pyrrolo[2,3-d]pyrimidin-4-yl)-I,6-diazaspiro[3.4]octan-1-yl]-3-oxopropanenitrile, and a pharmaceutical formulation thereof.

### FIELD OF THE INVENTION

The present invention relates to a new pharmaceutical use of Delgocitinib, 3-[(3S,4R)-3-methyl-6-(7H-pyrrolo[2,3-d]pyrimidin-4-yl)-1,6-diazaspiro[3.4]octan-1-yl]-3-oxopropanenitrile. In another aspect, the present invention relates to the treatment of frontal fibrosing alopecia (FFA).

### BACKGROUND OF THE INVENTION

Frontal fibrosing alopecia (FFA) was first described in 1994 and is a type of primary lymphocytic cicatricial alopecia. FFA is considered a clinical variant of lichen planopilaris (LPP) due to their shared histopathologic features. This variant of LPP is often found in postmenopausal women and is increasing in prevalence. FFA is characterized by the recession of the frontal, temporal, or frontotemporal hairline; the clinical pattern is distinct and usually includes eyebrow hair loss, as well as other associated symptoms. Pruritus, facial papules, eyelash loss, body hair involvement, and trichodynia may also occur in addition to the frontotemporal recession and eyebrow loss classically seen.

FFA is also reported in patients with hypothyroidism, contact allergy to fragrances, regular sunscreen use, and autoimmune diseases including lupus erythematosus and rheumatoid arthritis.

Early diagnosis and prompt treatment are critical as FFA is a progressive disorder that can result in permanent hair loss. However, even with aggressive therapy, FFA may progress. There are a limited number of published guidelines for the treatment of FFA and there is no consensus or standard treatment regimen for FFA.

Intralesional corticosteroids may achieve some disease stabilization, with associated adverse effects such as pain and skin atrophy, and topical corticosteroids are often not effective. In addition, no therapeutic interventions selectively target cellular or molecular key elements of FFA pathogenesis.

The etiology and pathogenesis of FFA are not completely understood and remain areas of investigation. The inflammatory cell infiltrate surrounding lesional hair follicles, namely around the bulge and infundibulum, is characterized by an increase in CD8+ GranzymeB+ cytotoxic T cells and plasmacytoid dendritic cells (pDC), with elevated levels of the chemokine receptor CXCR3 expression. The available evidence suggests a key role for interferon (IFN)-γ in inducing hair follicule immune privilege (HFIP) bulge collapse and subsequent immune-mediated epithelial hair follicle stem cells (eHFSC) destruction observed in FFA. Therapeutically, bulge immune privilege protection/restoration or neutralizing IFN-γ may help to better manage this highly treatment-resistant cicatricial alopecia.

Several JAK-inhibitors are in clinical development or are already on the market. Ruxolitinib, the first FDA approved JAK1 and JAK2 inhibitor, is an oral drug which is approved in several countries/regions for treatment of patients with myelofibrosis, and other various chronic inflammatory conditions. Other JAK-inhibitors, such as Tofacitinib and Baricitinib are also approved or under development for treatment of various chronic inflammatory conditions.

WO2011/013785 describes nitrogen-containing spirocyclic compounds and pharmaceutical uses thereof. The compounds are stated to be JAK kinase 3 inhibitors useful for the prevention or treatment of e.g. autoimmune diseases, allergic diseases, psoriasis, rheumatoid arthritis and atopic dermatitis.

EP 2813228 A1 describes the pharmaceutical use of JAK inhibitors, and more specifically a pharmaceutical composition for treating skin diseases such as senile xerosis, asteatosis, eczema and contact dermatitis.

WO2017/050891 describes the use of a pharmaceutical composition for treating alopecia areata.

Consequently, there is an unmet medical need for new treatment of FFA with high efficacy in combination with an attractive safety profile.

### SUMMARY OF THE INVENTION

The present invention relates to the treatment of FFA comprising the compound of formula (I)

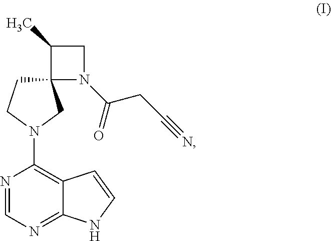 [SMILES: C[C@H]1CN(C(=O)CC#N)[C@]12CCN(c1ncnc3[nH]ccc13)C2]

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof.

In one aspect the present invention relates to the use of the compound of formula (I) for use in the treatment of FFA.

In another aspect, the present invention relates to the compound of formula (I) for use in the treatment of FFA. In yet another aspect the invention relates to the compound of formula (I) for use in topical treatment of FFA. In yet another aspect the topical formulation is a cream. In yet another aspect the present invention relates to the use of the compound of formula (I), which is administered twice daily. In yet another aspect the present invention relates to the use of the compound of formula (I), which is administered twice daily for 12 weeks. In yet another aspect, the present invention relates to the compound of formula (I) which is administered in a concentration of 20 mg/g.

In another aspect, the present invention relates to the use of the compound of formula (I) in the manufacture of a pharmaceutical composition for the treatment of FFA. In yet another aspect, the pharmaceutical composition is a topical formulation. In yet another aspect the topical formulation is a cream.

In another aspect, the present invention relates to a pharmaceutical composition comprising a compound of formula (I)

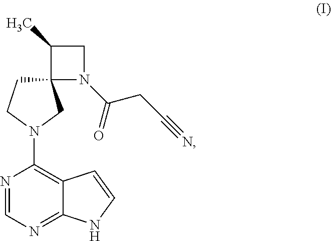 [SMILES: C[C@H]1CN(C(=O)CC#N)[C@]12CCN(c1ncnc3[nH]ccc13)C2]

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof, for the treatment of FFA.

In another aspect, the present invention relates to the pharmaceutical composition comprising the compound of formula (I) wherein the treatment is topical treatment, e.g. treatment with a cream.

In another aspect, the present invention relates to the pharmaceutical composition wherein the compound of formula (I) is administered as 20 mg/g concentration. In another aspect, the compound of formula (I) is administered as a twice daily application. In another aspect, the compound of formula (I) is administered as a twice daily application for 12 weeks.

In another aspect, the present invention relates to a method for treating FFA in a subject in need thereof, which method comprises the step of administering to said subject a therapeutically effective amount of the compound of formula (I).

In another aspect, the present invention relates to a method for treating FFA in a subject in need thereof, wherein the administration is topical. In another aspect, the present invention relates to a method for treating FFA in a subject in need thereof, wherein the topical formulation is a cream. In another aspect, the present invention relates to a method for treating FFA in a subject in need thereof, wherein the compound of formula (I) is administered in a concentration of 20 mg/g.

The present invention also provides for the use of 3-[(3S,4R)-3-methyl-6-(7H-pyrrolo[2,3-d]pyrimidin-4-yl)-1,6-diazaspiro[3,4]octan-1-yl]-3-oxopropanenitrile in the treatment of FFA.

The present invention further provides for the above use administered as a once daily application. The present invention further provides for the above use administered as a twice daily application. The present invention further provides for the above use administered in a concentration of 20 mg/g.

### DETAILED DESCRIPTION OF THE INVENTION

The compound of formula (I)

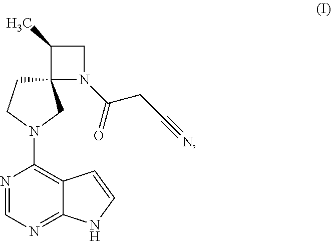 [SMILES: C[C@H]1CN(C(=O)CC#N)[C@]12CCN(c1ncnc3[nH]ccc13)C2]

3-[(3S,4R)-3-methyl-6-(7H-pyrrolo[2,3d]pyrimidin-4-yl)-1,6-diazaspiro[3,4]octan-1-yl]-3-oxopropanenitrile, was described previously in WO2011/013785 as a JAK 3 kinase inhibitor for the treatment of e.g. autoimmune diseases, allergic diseases, psoriasis, rheumatoid arthritis and atopic dermatitis, and in EP 2813228 A1 for treating skin diseases such as senile xerosis, asteatosis, eczema and contact dermatitis.

The compound of formula (I) can be produced according to the method described in Preparation 6 of WO2011/013785.

The “pharmaceutically acceptable salt” may be any non-toxic salts of the compound of formula (I), and includes a salt with an inorganic acid, a salt with an organic acid, a salt with an inorganic base, a salt with an organic base, and a salt with an amino acid.

The salt with an inorganic acid includes a salt with hydrochloric acid, nitric acid, sulphuric acid, phosphoric acid, hydrobromic acid, etc. The salt with an organic acid includes salt with oxalic acid, maleic acid, citric acid, fumaric acid, lactic acid, malic acid, succinic acid, tartaric acid, acetic acid, trifluoroacetic acid, citric acid monohydrate, gluconic acid, ascorbic acid, methanesulfonic acid, benzenesulfonic acid, p-toluenesulfonic acid, etc. The salt with an inorganic base includes sodium salt, potassium salt, calcium salt, magnesium salt, ammonium salt, etc. The salt with an organic base includes a salt with methylamine, diethylamine, trimethylamine, triethylamine, ethanolamine, diethanolamine, triethanolamine, ethylenediamine, tris(hydroxymethyl)methylamine, dicyclohexylamine, N,N′-dibenzylethylenediamine, guanidine, pyridine, picoline, choline, cinchonine, meglumine, etc. The salt with an amino acid includes a salt with lysine, arginine, aspartic acid, glutamic acid, etc.

According to well-known methods, each salt may be obtained by reacting the compound of formula (I) with an inorganic base, an organic base, an inorganic acid, an organic acid or an amino acid.

The compound of formula (I) may be labeled with isotopes, e.g. ³H, ¹⁴C, ³⁵S.

In one aspect, the compound of formula (I) or a pharmaceutically acceptable salt thereof is the substantially purified compound of formula (I) or a pharmaceutically acceptable salt thereof. In another aspect the compound of formula (I) or a pharmaceutically acceptable salt thereof is in the purity of 80% or more.

The “pharmaceutical composition” includes an oral preparation such as tablet, capsule, granule, powder, lozenge, syrup, emulsion and suspension, or a parenteral preparation such as topical agent, suppository, injection, eyedrop, nasal drug and pulmonary drug. In another aspect, the pharmaceutical composition is a topical formulation, such as ointment and cream. In yet another aspect, the pharmaceutical composition is a cream.

The pharmaceutical composition of the present invention is produced by appropriately mixing the compound of formula (I) or a pharmaceutically acceptable salt thereof with at least one type of pharmaceutically acceptable excipients or carriers in appropriate amounts according to methods known in the technical field of medicinal preparation.

The content of the compound of formula (I) or a pharmaceutically acceptable salt thereof in the pharmaceutical composition depends on its dosage forms, dosage amounts, etc., and for example, is 0.1 to 100% by weight of the entire composition. For example, the content is 0.10, 0.20, 0.25, 0.30, 0.50, 0.75, 0.8, 1.0, 1.25, 1.50, 1.75, 2.00, 2.25, 2.50, 2.75, 3.00, 3.25, 3.50, 3.75 or 4.00% by weight of the entire composition.

In one aspect, the content of the compound of formula (I) or a pharmaceutically acceptable salt thereof in the pharmaceutical composition is 0.1% by weight of the entire composition.

In one aspect, the content of the compound of formula (I) or a pharmaceutically acceptable salt thereof in the pharmaceutical composition is 0.3% by weight of the entire composition.

In one aspect, the content of the compound of formula (I) or a pharmaceutically acceptable salt thereof in the pharmaceutical composition is 0.8% by weight of the entire composition.

In one aspect, the content of the compound of formula (I) or a pharmaceutically acceptable salt thereof in the pharmaceutical composition is 2% by weight of the entire composition.

In one aspect, the content of the compound of formula (I) or a pharmaceutically acceptable salt thereof in the pharmaceutical composition is 3% by weight of the entire composition.

The term “therapeutically effective amount” of the compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject.

The “pharmaceutically acceptable excipient” or “pharmaceutically acceptable carrier” includes various conventional organic or inorganic excipient or carrier substances for pharmaceutical materials, e.g. a disintegrant, a binder, a fluidizer, a lubricant, a solvent, a solubilizing agent, a suspending agent, a tonicity agent, a buffer, and a soothing agent for liquid preparations, and a base, an emulsifier, a surfactant, a humectant, a stabilizer, a stabilizing agent, a dispersant, a plasticizer, a pH regulator, an absorption promoter, a gelling agent, an antiseptic, a filler, a resolvent. Further, an additive including a preserving agent, a preservative, an antioxidant, and a colorant may be used, if needed.

The disintegrant includes carmellose, carmellose calcium, carmellose sodium, sodium carboxymethyl starch, croscarmellose sodium, crospovidone, low-substituted hydroxypropyl cellulose, hydroxypropyl methylcellulose, crystalline cellulose, etc.

The binder includes hydroxypropyl cellulose, hydroxypropyl methylcellulose, povidone, crystalline cellulose, white soft sugar, dextrin, starch, gelatin, carmellose sodium, gum arabic, etc.

The fluidizer includes light anhydrous silicic acid, magnesium stearate, etc.

The lubricant includes magnesium stearate, calcium stearate, talc, etc.

The solvent includes purified water, ethanol, propylene glycol, macrogol, sesame oil, corn oil, olive oil, medium chain triglyceride, etc.

The solubilizing agent includes propylene glycol, D-mannitol, benzylbenzoate, ethanol, triethanolamine, sodium carbonate, sodium citrate, etc.

The suspending agent includes benzalkonium chloride, carmellose, hydroxypropyl cellulose, propylene glycol, povidone, methyl cellulose, glyceryl monostearate, etc.

The tonicity agent includes glucose, D-sorbitol, sodium chloride, D-mannitol, etc.

The buffer or pH regulator includes phosphate or citrate salts, sodium hydrogen phosphate, sodium acetate, sodium carbonate, sodium citrate, sodium citrate dihydrate, citric acid monohydrate, hydrochloric acid, sodium hydroxide, etc.

The base includes water, animal or plant oils (e.g., olive oil, corn oil, peanut oil, sesame oil, castor oil, safflower oil, etc.), lower alcohols (e.g., ethanol, propanol, propylene glycol, 1,3-butylene glycol, phenol, etc.), higher fatty acids and esters thereof, waxes, higher alcohols, polyhydricalcohols, hydrocarbons (e.g., white soft paraffin, liquid paraffin, paraffin, hard paraffin, etc.), hydrophilic petrolatum, purified lanolin, absorptive ointment, hydrous lanolin, hydrophilic ointment, starch, pullulan, polydimethylsiloxane, isopropyl myristate, gum arabic, tragacanth gum, gelatin, dextran, cellulose derivatives (e.g., methyl cellulose, carboxymethyl cellulose, hydroxyethyl cellulose), polymers (e.g. carboxyvinyl polymer, sodium polyacrylate, polyvinyl alcohol, polyvinyl pyrrolidone, etc.), propylene glycol, macrogol (e.g., macrogol 200 to 600, etc.), and combinations of two or more kinds of these. In one aspect, the base is liquid paraffin.

The emulsifier or surfactant includes mixtures of fatty acids, especially cetyl alcohol, stearyl alcohol, and cetostearyl alcohol, macrogol cetostearyl ether, sorbitan esters, sucrose esters, ect.

The stabilizer includes sucrose esters, other sorbitan esters and poly sorbates, glyceol, propylene glycol, ethanol, cetostearyl alcohol, ect.

The acidifying agent includes strong acid selected from e.g. hydrochloric acid or citric acid.

The chelating agent includes ethylenediaminetetraacetic acid (EDTA), disodium edetate, ethylene glycol tetraacetic acid (EGTA), ethylene diamine, phosphoric acid, ect.

The preservative includes ethyl parahydroxybenzoate, chlorobutanol, benzyl alcohol, sodium dehydroacetate, sorbic acid, chlorocresol, dichlorobenzyl alcohol, glycerol, ethanol, propylene glycol, benzoic acid/sodium benzoate, diazolidiny urea, benzalkonium chloride, etc.

The antioxidant includes sodium sulfite, ascorbic acid, disodium edetate, trisodium edetate, alphatocopherol, butylhydroxy anisole, etc.

The colorant includes food dye, β-carotene, etc.

The pharmaceutical composition of the present invention may be administered to a mammal, such as a human being. A dosage amount depends on subjects, diseases, symptoms, dosages forms, administration routes, etc., and may be in the range from about 0.01 mg to about 1 g, e.g. from about 0.01 mg to about 600 mg per day in terms of the content of the compound of formula (I) as an active ingredient. The dose may be administered at a time or in several divided doses.

A topical agent may be applied, for example, by application, inunction or spraying depending on the dosage form, etc. An application amount of the topical agent to the affected area can be selected depending on the content of the active ingredient, etc., and the topical agent can be applied, for example, at a time or in several divided amounts per day. The preferable application is once daily or twice daily.

The phrase “JAK” refers to one or more enzymes of JAK1, JAK2, JAK3, and TYK2 belonging to JAK family.

The phrase “inhibit JAK” refers to inhibiting functions of JAK to disappear or attenuate its activity and inhibiting one or more enzymes belonging to JAK family. In one aspect, the phrase “inhibit JAK” refers to “inhibit human JAK”. The inhibition of functions or the disappearance or attenuation of the activity is, in one aspect, conducted in the situations of human clinical application.

The “JAK inhibitor” may be any substances which inhibit JAK, and may be, for example, low-molecular weight compounds, nucleic acid, polypeptide, protein, antibody, vaccine, etc. In one aspect, the “JAK inhibitor” is a “human JAK inhibitor”. In another aspect, the JAK inhibitor is the compound of formula (I) or a pharmaceutically acceptable salt thereof. In yet another aspect, the JAK inhibitor is the compound of formula (I).

The term “treatment” used herein includes amelioration of a symptom, prevention of an aggravation, maintenance of a remission, prevention of an exacerbation, and prevention of a recurrence. The term prevention refers to suppressing occurrence of a symptom.

The term “treatment” may also include the delaying of the progression of the disease, disorder or condition, the amelioration, alleviation or relief of symptoms and complications, and/or cure or elimination of the disease, disorder or condition.

The term treatment may also mean the management and care of a patient for the purpose of combating the disease, condition or disorder.

The terms “disease”, “disorder” and “condition” as used herein are used interchange-ably to specify a state of a patient which is not the normal physiological state of a human being.

An embodiment of the present invention includes a pharmaceutical composition for treating or preventing cicatricial alopecia, comprising 3-[(3S,4R)-3-methyl-6-(7H-pyrrolo[2,3-d]pyrimidin-4-yl)-1,6-diazaspiro[3.4]octan-1-yl]-3-oxopropanenitrile and a pharmaceutically acceptable excipient or carrier. Another embodiment of the present invention includes a pharmaceutical composition for treating or preventing LPP, comprising 3-[(3S,4R)-3-methyl-6-(7H-pyrrolo[2,3-d]pyrimidin-4-yl)-1,6-diazaspiro[3.4]octan-1-yl]-3-oxopropanenitrile and a pharmaceutically acceptable excipient or carrier. Another embodiment of the present invention includes a pharmaceutical composition for treating or preventing FFA, comprising 3-[(3S,4R)-3-methyl-6-(7H-pyrrolo[2,3-d]pyrimidin-4-yl)-1,6-diazaspiro[3.4]octan-1-yl]-3-oxopropanenitrile and a pharmaceutically acceptable excipient or carrier.

An embodiment of the present invention includes a use of 3-[(3S,4R)-3-methyl-6-(7H-pyrrolo[2,3-d]pyrimidin-4-yl)-1,6-diazaspiro[3.4]octan-1-yl]-3-oxopropanenitrile for treating or preventing cicatricial alopecia. Another embodiment of the present invention includes a use of 3-[(3S,4R)-3-methyl-6-(7H-pyrrolo[2,3-d]pyrimidin-4-yl)-1,6-diazaspiro[3.4]octan-1-yl]-3-oxopropanenitrile for treating or preventing LPP.

Another embodiment of the present invention includes a use of 3-[(3S,4R)-3-methyl-6-(7H-pyrrolo[2,3-d]pyrimidin-4-yl)-1,6-diazaspiro[3.4]octan-1-yl]-3-oxopropanenitrile for treating or preventing FFA.

An embodiment of the present invention includes a method for treating or preventing cicatricial alopecia characterized by administering to a mammal a therapeutically effective amount of 3-[(3S,4R)-3-methyl-6-(7H-pyrrolo[2,3-d]pyrimidin-4-yl)-1,6-diazaspiro[3.4]octan-1-yl]-3-oxopropanenitrile. Another embodiment of the present invention includes a method for treating or preventing LPP characterized by administering to a mammal a therapeutically effective amount of 3-[(3S,4R)-3-methyl-6-(7H-pyrrolo[2,3-d]pyrimidin-4-yl)-1,6-diazaspiro[3.4]octan-1-yl]-3-oxopropanenitrile. Another embodiment of the present invention includes a method for treating or preventing FFA characterized by administering to a mammal a therapeutically effective amount of 3-[(3S,4R)-3-methyl-6-(7H-pyrrolo[2,3-d]pyrimidin-4-yl)-1,6-diazaspiro-[3.4]octan-1-yl]-3-oxopropanenitrile.

In one aspect the mammal is a human being.

In another aspect, the human being is a person suffering from a disease who is in need of medical care.

In another aspect, the disease is cicatricial alopecia. In another aspect, the disease is LPP. In another aspect, the disease is FFA.

In another aspect, the present invention relates to a therapeutic or preventive agent for cicatricial alopecia comprising the compound of formula (I). In another aspect, the present invention relates to a therapeutic or preventive agent for LPP comprising the compound of formula (I). In another aspect, the present invention relates to a therapeutic or preventive agent for FFA comprising the compound of formula (I).

In another aspect, the present invention relates to a pharmaceutical composition, comprising the compound of formula (I) and one or more of a pharmaceutically acceptable excipient or carrier.

In another aspect, the present invention relates to a pharmaceutical formulation for topical administration, comprising the compound of formula (I) and one or more of a pharmaceutically acceptable excipient.

In another aspect, the present invention relates to a pharmaceutical formulation which is a cream, comprising the compound of formula (I) and one or more of a pharmaceutically acceptable excipient.

In another aspect, a pharmaceutically acceptable excipient could be one or more of a base selected from medium chain triglycerides, safflower oil, castor oil, liquid paraffin or mixtures thereof. For example the base could be liquid paraffin.

The base could be present at various amounts from about 50 mg/g to about 500 mg/g of liquid paraffin, e.g. from about 75 mg/g to about 300 mg/g, and e.g. 100 mg/g.

In another aspect, a pharmaceutically acceptable surfactant, emulsifier or stabilizer could be one or more selected from cetyl alcohol, stearyl alcohol, cetostearyl alcohol or mixtures thereof. For example the surfactant, emulsifier or stabilizer could be cetostearyl alcohol.

The surfactant, emulsifier or stabilizer could be present at various amounts from about 20 mg/g to about 100 mg/g of cetostearyl alcohol, e.g. from about 40 mg/g to about 80 mg/g, and e.g. 72 mg/g.

In another aspect, the pharmaceutically acceptable surfactant, emulsifier or stabilizer could be one or more selected from sorbitan esters, sucrose esters, macrogol cetostearyl ether or mixtures thereof. For example the surfactant or emulsifier could be macrogol cetostearyl ether.

The surfactant, emulsifier or stabilizer could be present at various amounts from about 9 mg/g to about 25 mg/g of macrogol cetostearyl ether, e.g. from about 15 mg/g to about 20 mg/g, and e.g. 18 mg/g.

In another aspect, a pharmaceutically acceptable buffer or pH regulator could be one or more of a phosphate or citrate salt, sodium acetate, sodium carbonate, sodium citrate dihydrate, hydrochloric acid or mixtures thereof. For example the buffer or pH regulator could be one or more of citric acid monohydrate and sodium citrate dihydrate.

The buffer or pH regulator could be present at various amounts from about 0.5 mg/g to about 4 mg/g of citric acid monohydrate, e.g. from about 0.7 mg/g to about 2 mg/g, and e.g. 1 mg/g.

The buffer or pH regulator could be present at various amounts from 0 mg/g to about 1 mg/g of sodium citrate dihydrate, e.g. from 0 mg/g to about 0.5 mg/g, and e.g. absent.

In another aspect, a pharmaceutically acceptable preservative could be one or more selected from benzyl alcohol, sodium dehydroacetate, sorbic acid/salts or mixtures thereof. For example the preservative could be benzyl alcohol.

The preservative could be present at various amounts from about 7 mg/g to about 13 mg/g of benzyl alcohol, e.g. from about 9 mg/g to about 11 mg/g, and e.g. 10 mg/g.

In another aspect, a pharmaceutically acceptable antioxidant could be one or more selected from sodium sulphite, disodium edetate, trisodium edetate, butylhydroxy anisole or mixtures thereof. For example the antioxidant could be butylhydroxy anisole.

The antioxidant could be present at various amounts from about 0.05 mg/g to about 0.3 mg/g of butylhydroxy anisole; e.g. from about 0.1 mg/g to about 0.25 mg/g, and e.g. 0.2 mg/g.

In another aspect, a pharmaceutically acceptable chelating agent could be one or more of EDTA, disodium edetate, EGTA, or ethylene diamine. For example the chelating agent could be disodium edetate.

The chelating agent could be present at various amounts from about 0.05 mg/g to about 1.5 mg/g of disodium edetate, e.g. from about 0.5 mg/g to about 1 mg/g, and e.g 0.6 mg/g.

In another aspect, a pharmaceutically acceptable acidifying agent could be one or more of a strong acid, e.g. hydrochloric acid or citric acid. For example the acidifying agent could be hydrochloric acid.

The acidifying agent could be present at various amounts from 0 mg/g to about 25 mg/g of hydrochloric acid, e.g. from about 10 mg/g to about 20 mg/g, and e.g. 17.7 mg/g.

In another aspect, a pharmaceutically acceptable solvent could be purified water, which could be present at various amounts from about 500 mg/g to about 900 mg/, and e.g. 760 mg/g.

The present pharmaceutical formulations can be prepared according to the method described in WO 2020/229622.

Exemplary specific pharmaceutical formulations of the present invention are:

Delgocitinib 20 mg/g; paraffin liquid 100 mg/g; cetostearyl alcohol 72 mg/g; macrogol cetostearyl ether 18 mg/g; benzyl alcohol 10 mg/g; citric acid monohydrate 1.0 mg/g; butylhydroxy anisole 0.2 mg/g; disodium edetate 0.6 mg/g; 3M hydrochloric acid 17.7 mg/g, and purified water up to 1 g.

Delgocitinib 20 mg/g; paraffin liquid 100 mg/g; cetostearyl alcohol 72 mg/g; macrogol cetostearyl ether 18 mg/g; benzyl alcohol 10 mg/g; citric acid monohydrate 1.0 mg/g; Sodium citrate 1 mg/g; disodium edetate 1.3 mg/g; hydrochloric acid 4.99 mg/g; butylhydroxy anisole 0.2 mg/g; and purified water up to 1 g.

The present invention describes its effect in topical treatment of cicatricial alopecia. In another aspect, the present invention describes its effect in topical treatment of LPP.

In another aspect, the present invention describes its effect in topical treatment of FFA.

The present invention provides a clinical trial to assess the efficacy and safety of delgocitinib cream 20 mg/g in adults subjects with FFA.

In the present trial, the molecular signature changes following topical application of delgocitinib cream 20 mg/g in subjects with FFA will be evaluated. The safety and preliminary efficacy of delgocitinib cream in subjects with FFA will also be evaluated.

Primary objective: To assess molecular signature changes following topical application of delgocitinib cream 20 mg/g in subjects with FFA.

Secondary objective: To evaluate the safety and tolerability of delgocitinib cream 20 mg/g following topical application in subjects with FFA during the vehicle-controlled treatment period.

Exploratory objective: To evaluate the safety and tolerability of delgocitinib cream 20 mg/g during the open-label extension. To evaluate the preliminary efficacy of delgocitinib cream 20 mg/g following topical application in subjects with FFA.

### EXAMPLES

### Trial Design:

A Phase 2a, randomized, double-blind, vehicle-controlled, single-site, exploratory trial of delgocitinib cream 20 mg/g in subjects with FFA.

The trial will consist of 2 cohorts: Cohort 1 will include approximately 30 subjects with FFA and Cohort 2 will include approximately 5 healthy postmenopausal female subjects.

### Cohort 1:

All subjects will read and sign an informed consent form prior to any trial-related activities being performed. Subjects who fulfill all of the inclusion criteria and none of the exclusion criteria will be randomized into the trial. After a screening period of no more than 30 days, eligible subjects will be randomized (1:1) on Day 1 to apply delgocitinib cream 20 mg/g or vehicle cream twice daily for 12 weeks during the vehicle-controlled treatment period. All subjects who complete the vehicle controlled treatment period will then continue into a open-label extension period and apply delgocitinib cream 20 mg/g twice daily for 12 weeks. The open-label extension period will be followed by a 2-week safety follow-up period. For scheduled trial visits, subjects will come to the site on 7 occasions: screening, Day 1, Week 4, Week 8, Week 12, Week 24, and Week 26/early termination. Subjects will also be contacted by phone on 2 occasions: at Week 16 and at Week 20.

On Day 1, a lesional target area on the scalp (eg, frontal, periauricular, or temporal area) will be identified. The lesional target area should be large enough to accommodate the hair count/trichoscopy assessment and the collection of all skin samples (ie, skin biopsies, tape stripping, and skin swabs) and should be treated with the investigational medicinal product. If needed, a second target area could be selected to allow for skin samples collection.

### Cohort 2:

All subjects will read and sign an informed consent form prior to any trial-related activities being performed. Subjects who fulfill all of the inclusion criteria and none of the exclusion criteria will be accepted into the trial. After a screening period of no more than 30 days, eligible subjects will be asked to come at the site for skin samples collection. Screening and skin samples collection on Day 1 can be performed on the same day (if subjects do not have a washout period) or on two separate visits, at the investigator's discretion. No investigational product will be administered in Cohort 2. For scheduled trial visits, subjects will come to the site on up to 3 occasions: screening, Day 1, and optional follow-up visit.

### Inclusion Criteria

In order to be eligible to participate in this trial, a subject must meet all of the following criteria, either at the screening and Day 1 visits or only at one of the specified visits (screening or Day 1) as noted in the criterion:

### Inclusion Criteria for all Subjects:

Subject is willing to participate and is capable of giving informed consent. Note: Consent must be obtained prior to any trial-related procedures.
Subjects must be willing to comply with all trial procedures and must be available for the duration of the trial.
   Inclusion Criteria for Cohort 1 Only (Subjects with FFA):
Male or female subject aged 18 years of age or older at the time of consent.
Female subject of childbearing potential has had a negative serum pregnancy test at screening and negative urine pregnancy test at Day 1.
Subject has clinically confirmed diagnosis of FFA based on investigator' judgment.
Subject has a target area with a perifollicular erythema score 2 and a perifollicular scale score 2 at Screening and Day 1.
For subject who uses make-up, moisturizers, creams, lotions, cleansers, and/or sunscreens on the face, subject has used the same product brands/types for a minimum period of 4 weeks prior to Day 1, agrees not to change brand/type or frequency of use throughout the trial, agrees not to apply those products on the treated area during the trial, and agrees not to use make-up, moisturizers, creams, lotions, cleansers, and/or sunscreens on the face on the clinic visit days before the visit.
Subject is willing to maintain a consistent hair style and hair style regimen, including shampoo and hair products (including hair dye, process, and timing to hair appointments), and to refrain from weaves or extensions throughout the course of the trial and for 4 weeks prior to Day 1. Note: Hair dying and shaving of scalp is allowed during the trial but not within 48 hours prior to a trial visit.
For female subject of childbearing potential involved in any sexual intercourse that could lead to pregnancy: the subject must agree to use an effective contraceptive method from at least 4 weeks prior to Day 1 until at least 4 weeks after the last investigational medicinal product application.

### Inclusion Criteria for Cohort 2 Only (Healthy Subjects):

Female subject aged 45 years of age or older at the time of consent.
Female is postmenopausal as defined as follows: —Female subject who has had surgical sterilization (hysterectomy, bilateral oophorectomy, or bilateral salpingectomy); —Female subject who has had a cessation of menses for at least 12 months prior to the screening visit without an alternative medical cause.
Subject is in good general health, according to the investigator's judgment based on vital signs, medical history, and brief physical examination

### Exclusion Criteria

A subject who meets any of the following criteria at the screening and/or Day 1 visits, as applicable, will be excluded from participation in this trial:

### Exclusion Criteria for all Subjects:

Subject is a female who is breastfeeding, pregnant, or who is planning to become pregnant during the trial.
Subject has received any marketed or investigational biological agent within 12 weeks or 5 half lives (whichever is longer) prior to Day 1.
Subject is currently receiving a nonbiological investigational product or device or has received one within 4 weeks prior to Day 1.
Subject has had excessive sun exposure or has used tanning booths within 4 weeks prior to Day 1 or is not willing to minimize natural and artificial sunlight exposure during the trial. Use of sunscreen products (except on treated areas for subjects in Cohort 1) and protective apparel are recommended when sun exposure cannot be avoided.
Subject has a history of an allergic reaction or significant sensitivity to lidocaine or other local anesthetics.
Subject has a history of hypertrophic scarring or keloid formation in scars or suture sites.
   Exclusion Criteria for Cohort 1 Only (Subjects with FFA):
History of other scalp/hair disease including discoid lupus erythematosus and central centrifugal cicatricial alopecia.
Presence of active dermatologic condition that might interfere with FFA diagnosis and/or interfere with the trial assessments.
Subject who has undergone scalp reduction surgery or hair transplantation.
Use of adhesive wigs during the trial.
Subject is known to have immune deficiency or is immunocompromised.
Subject has a history of cancer or lymphoproliferative disease within 5 years prior to Day 1. Subjects with successfully treated nonmetastatic cutaneous squamous cell or basal cell carcinoma and/or localized carcinoma in situ of the cervix are not to be excluded.
Subject had a major surgery within 8 weeks prior to Day 1 or has a major surgery planned during the trial.
Subject has any clinically significant medical condition or physical/laboratory/ECG/-vital signs abnormality that would, in the opinion of the investigator, put the subject at undue risk or interfere with interpretation of trial results.
Subject has positive results for hepatitis B surface antigens (HBsAg), antibodies to hepatitis B core antigens (anti-HBc), hepatitis C virus (HCV), or human immune-deficiency virus (HIV).
Subject has used treatment with agents (including natural products or nutritional supplement such as Viviscal, Nutrafol, and/or biotin) that may affect hair regrowth in the last 4 weeks prior to Day 1.
Subject has used intralesional scalp corticosteroids or platelet rich plasma injection in the last 4 weeks prior to Day 1.
Subject has used systemic treatment with immunosuppressive/modulating medication or medication that could affect FFA (eg, corticosteroids, methotrexate, minoxidil, hydroxychloroquine, retinoids, calcineurin inhibitor, tetracyclines, pioglitazone, spironolactone, or 5 α reductase inhibitors) within 4 weeks prior to Day 1. Note: Intranasal corticosteroids and inhaled corticosteroids are allowed. Eye and ear drops containing corticosteroids are also allowed. Note: Standard doses of systemic antihistamines are allowed.
Subject has used any topical medicated treatment that could affect FFA within 2 weeks prior to Day 1, including, but not limited to, topical corticosteroids, calcineurin inhibitors, minoxidil, phosphodiesterase-4 (PDE-4) inhibitors.
Subjects has received treatment with JAK inhibitors (systemic or topical) within 4 weeks prior to Day 1.
Subject has received any ultraviolet (UV)-B phototherapy (including tanning beds), excimer laser, or any other phototherapy within 4 weeks prior to Day 1.
Subject has had psoralen-UV-A (PUVA) treatment within 4 weeks prior to Day 1.
Subject has a known or suspected allergy to delgocitinib or any component of the investigational product.
Subject has a history of an allergic reaction or significant sensitivity to hypoallergenic ink.
Subject has a known history of clinically significant drug or alcohol abuse in the last year prior to Day 1.

### Exclusion Criteria for Cohort 2 Only (Healthy Subjects):

Subject has a history of skin disease or presence of skin condition that, in the opinion of the investigator, would interfere with the trial assessments.
Subject has any clinically significant medical condition or physical/vital signs abnormality that would, in the opinion of the investigator, put the subject at undue risk or interfere with interpretation of trial results.
Subject has a known history of chronic infectious disease (e.g., hepatitis B, hepatitis C, or HIV).
Subject has used a topical medicated treatment on the targeted skin sites within 2 weeks prior to skin samples collection (Day 1).

### Efficacy Assessments (Cohort 1 Only)

Clinical evaluations of FFA will be performed by an experienced and qualified dermatologist (board certified or equivalent) or other suitably qualified. To assure consistency and reduce variability, the same assessor should perform all assessments on a given subject whenever possible.

### Lichen Planopilaris Activity Index

The LPPAI will be assessed at the visits. It is a quantitative measure of disease activity. The LPPAI records symptoms (pruritus, pain, burning), signs (erythema, perifollicular erythema and scale), a measure of activity (the anagen pull test), and spreading of the condition. These subjective and objective measures have been assigned a numeric value to establish a disease activity score. The weights given to the symptoms (30%), signs (30%), anagen pull test (25%), and presence of spreading (15%) led to the equation: LPPAI (0-10)=(pruritus+pain+burning)/3+(scalp erythema+perifollicular erythema+perifollicular scale)/3+2.5 (pull test)+1.5 (spreading/2). Symptoms and signs are recorded on a 4-point scale: 0=absent (negative), 1=mild (+/−), 2=moderate (+), and 3=severe (++, +++). The anagen pull test, when present, is a reliable measure of local disease activity. It involves taking hold of 10 to 20 hairs between the thumb, second and third fingers at the scalp end of the hair shafts, and pulling firmly away from the scalp with a perpendicular force to slide the fingers to the ends of the hair. The result is recorded both as a binary value (0 for no anagen hairs and 1 for the presence of anagen hairs) and as anagen hairs/total hairs pulled. Last is the assessment of disease extension, recorded as 0 (no spreading) versus 1 (indeterminate) versus 2 (spreading). When the hair loss is difficult to judge, the issue of extension is recorded as indeterminate. A detailed procedure of LPPAI score calculation is provided in Table 1

TABLE 1
Lichen Planopilaris Activity Index (LPPAI)

Date of visit
LPPAI (see below)
SCALE: 0-3
A Pruritus
B Pain
C Burning
D Erythema
E Perifollic erythema
F Perifollicular scale
Crusting
Pustules
Pull test: Anagen/Total
(0 = −, 1 = +)
Spreading?
no (0) ? (1) yes (2)
LPPAI (0-10) = (A + B + C + D + E + F)/3 + 2.5(pull test) + 1.5(spread/2)
Scale:
0 = negative
1 = +/−
2 = +
3 = ++, +++

### Frontal Fibrosing Alopecia Severity Score

The FFASS will be assessed at the visits. This index is based on the evaluation of the relevant clinical features in FFA. Those features are the grade of frontal and temporal hairline recession (from 1 to 5), grade of eyebrow loss (none, partial, or total), severity and extent of perifollicular erythema and hyperkeratosis, and severity and frequency of pruritus and pain associated with FFA. The resulting severity scores range from 0 to 25, with higher scores indicating greater FFA severity. The clinical features included in the FFASS are grouped into two categories: extent of alopecia (up to 21 points) and inflammation (up to 4 points). A detailed procedure of FFASS score calculation is provided in Table 2

TABLE 2
Frontal Fibrosing Alopecia Severity Score

PUNCTUATION

CLINICAL SIGNS

| 1. HAIRLINE RECESSION | | | |
|---|---|---|---|
| (measurement of band-like scarring area) | | | |
| Frontal (x2) | <1 | cm (1) | _{——}/10 |
| Temporal left | 1-2.99 | cm (2) | _{——}/5 |
| Temporal right | 3-4.99 | cm (3) | _{——}/5 |
| | 5-6.99 | cm (4) | |
| | >7 | cm (5) | |

2. LOSS OF EYEBROWS

| | |
|---|---|
| No (0)/Partial (0.5)/Total (1) | _{——}/1 |
| EXTENT OF ALOPECIA SCORE | _{——}/21 |

3. PERIFOLLICULAR INFLAMMATION
A) SEVERITY

| | | | |
|---|---|---|---|
| Erythema | No/Mild/Severe | 0/0.1/0.2 | _{——}/0.2 |
| Hyperkeratosis | | 0/0.5/1 | _{——}/1 |

B) EXTENT (along the frontotemporal hairline)

| | | | |
|---|---|---|---|
| Erythema | <25%/25-75%/>75% | 0/0.1/0.2 | _{——}/0.2 |
| Hyperkeratosis | | 0/0.5/1 | _{——}/1 |

ASSOCIATED SYMPTOMS
1. PRURITUS

| | | |
|---|---|---|
| Severity | No (0) | _{——}/0.2 |
| | Mild/Occasional (0.1) | _{——}/0.2 |
| Frequency | Severe/Daily (0.2) | |

2. PAIN

| | | |
|---|---|---|
| Severity | No (0) | _{——}/0.6 |
| Frequency | Mild/Occasional (0.3) | _{——}/0.6 |
| | Severe/Daily (0.6) | |

| | |
|---|---|
| GRADE OF INFLAMMATION SCORE | _{——}/4 |
| TOTAL FFASS SCORE | _{——}/25 |

### Perifollicular Erythema and Scale

The perifollicular erythema and perifollicular scale of the selected lesional target area will be assessed visually at the visits. Each clinical finding (ie, perifollicular erythema and perifollicular scale) will be scored using the 4-point severity scale presented in the Table 3. To be eligible for this trial, subjects must have a lesional target area with a perifollicular erythema score ≥2 and a perifollicular scale score ≥2 at Screening and Day 1.

TABLE 3
Perifollicular Erythema and Perifollicular Scale Severity Scale

| Score | Description |
|---|---|
| 0 | Absent/None |
| 1 | Mild |
| 2 | Moderate |
| 3 | Severe/Intense |

### Pruritus Numerical Rating Scale

The intensity of pruritus due to FFA will be recorded using a numerical rating scale. It will be assessed daily starting approximately 7 days prior to Day 1 and up to Day 8. Thereafter, it will be assessed at the visits This will be evaluated by asking subjects to assign a numerical score representing of the worst intensity over the last 24 hours of their symptoms on a scale from 0 to 10, with 0 indicating no symptoms and 10 indicating the worst imaginable symptoms.

### Burning Sensation Numerical Rating Scale

The intensity of burning sensation due to FFA will be recorded using an numerical rating scale. It will be assessed daily starting approximately 7 days prior to Day 1 and up to Day 8. Thereafter, it will be assessed at the visits. This will be evaluated by asking subjects to assign a numerical score representing of the worst intensity over the last 24 hours of their symptoms on a scale from 0 to 10, with 0 indicating no symptoms and 10 indicating the worst imaginable symptoms.

### Pain Numerical Rating Scale

The intensity of pain due to FFA will be recorded using an numerical rating scale. It will be assessed daily starting approximately 7 days prior to Day 1 and up to Day 8. Thereafter, it will be assessed at the visits. This will be evaluated by asking subjects to assign a numerical score representing of the worst intensity over the last 24 hours of their symptoms on a scale from 0 to 10, with 0 indicating no symptoms and 10 indicating the worst imaginable symptoms.

### Hair Counts/Trichoscopy

Hair counts/trichoscopy will be performed at the visits. Trichoscopy should be performed on the lesional target area, few centimeters away from the site of skin microbiome collection. The number of hairs, hair diameter, and hair density will be measured via fotofinder trichovision.

### Hair Line Measurements

Hair line measurements will be performed at the visits. Lateral canthus (right and left) to hairline, lower glabellar crease to hair line, top of frontalis muscle to hair line, and mid brow to hair line (right and left) will be measured using a disposable paper ruler.

### CLAUSES

In view of the description the present invention has in particular provided:

Clause 1. A compound of general formula (I)

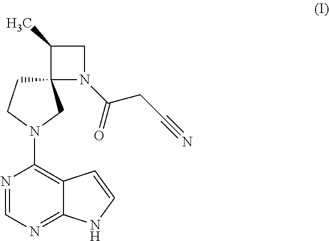

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof, for use in the treatment of cicatricial alopecia.

Clause 2. A compound of general formula (I)

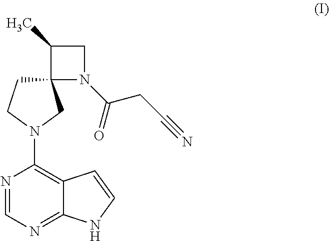

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof, for use in the treatment of LPP.

Clause 3. A compound of general formula (I)

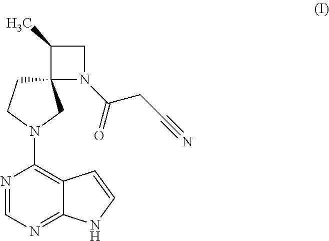

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof, for use in the treatment of FAA.

Clause 4. The compound for use according to any of the one of the preceding clauses wherein the treatment is topical treatment.

Clause 5. The compound for use according to any one of the preceding clauses wherein the compound of formula (I) is administered as a cream.

Clause 6. The compound for use according to any one of the preceding clauses wherein the compound of formula (I) is administered as 20 mg/g concentration.

Clause 7. The compound for use according to any one of the preceding clauses wherein the compound of formula (I) is administered as a twice daily application.

Clause 8. The compound for use according to any one of the preceding clauses wherein the compound of formula (I) is administered for 12 weeks.

Clause 9. A use of the compound of formula (I) in the manufacture of a pharmaceutical composition for the treatment of cicatricial alopecia.

Clause 10. A use of the compound of formula (I) in the manufacture of a pharmaceutical composition for the treatment of LPP.

Clause 11. A use of the compound of formula (I) in the manufacture of a pharmaceutical composition for the treatment of FFA.

Clause 12. The use according to any one of the preceding clauses 9-11 wherein the treatment is topical treatment.

Clause 13. The use according to any one of the preceding clauses 9-12 wherein the pharmaceutical composition is as a cream.

Clause 14. The use according to any one of the preceding clauses 9-13 wherein the compound of formula (I) is administered as 20 mg/g concentration.

Clause 15. The use according to any one of the preceding clauses 9-14 wherein the compound of formula (I) is administered as a twice daily application.

Clause 16. The use according to any one of the preceding clauses 9-15 wherein the compound of formula (I) is administered for 12 weeks.

Clause 17. A pharmaceutical composition comprising a compound of formula (I)

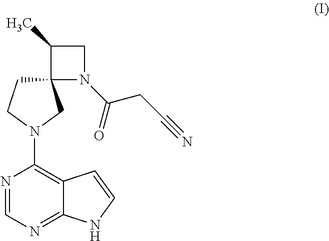 [SMILES: C[C@H]1CN(C(=O)CC#N)[C@]12CCN(c1ncnc3[nH]ccc13)C2]

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof, for the treatment of cicatricial alopecia.

Clause 18. A pharmaceutical composition comprising a compound of formula (I)

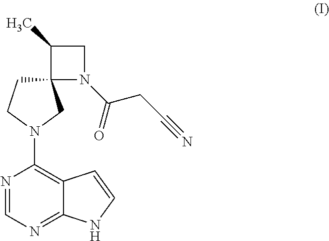 [SMILES: C[C@H]1CN(C(=O)CC#N)[C@]12CCN(c1ncnc3[nH]ccc13)C2]

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof, for the treatment of LPP.

Clause 19. A pharmaceutical composition comprising a compound of formula (I)

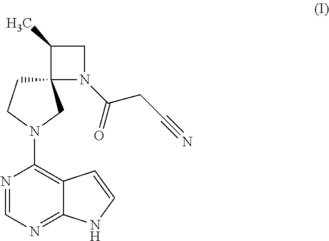 [SMILES: C[C@H]1CN(C(=O)CC#N)[C@]12CCN(c1ncnc3[nH]ccc13)C2]

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof, for the treatment of FFA.

Clause 20. The pharmaceutical composition according to any one of the preceding clauses 17-19 wherein the treatment is topical treatment.

Clause 21. The pharmaceutical composition according to any one of the preceding clauses 17-20 which is as a cream.

Clause 22. The pharmaceutical composition according to any one of the preceding clauses 17-21 wherein the compound of formula (I) is administered as 20 mg/g concentration.

Clause 23. The pharmaceutical composition according to any one of the preceding clauses 17-22 wherein the compound of formula (I) is administered as a twice daily application.

Clause 24. The pharmaceutical composition according to any one of the preceding clauses 17-23 wherein the compound of formula (I) is administered for 12 weeks.

Clause 25. A therapeutic or preventive agent for cicatricial alopecia, comprising a compound of formula (I)

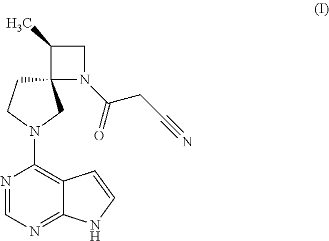 [SMILES: C[C@H]1CN(C(=O)CC#N)[C@]12CCN(c1ncnc3[nH]ccc13)C2]

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof as an active ingredient.

Clause 26. A therapeutic or preventive agent for LPP, comprising a compound of formula (I)

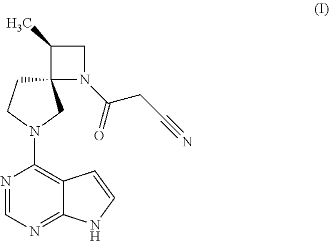 [SMILES: C[C@H]1CN(C(=O)CC#N)[C@]12CCN(c1ncnc3[nH]ccc13)C2]

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof as an active ingredient.

Clause 27. A therapeutic or preventive agent for FFA comprising a compound of formula (I)

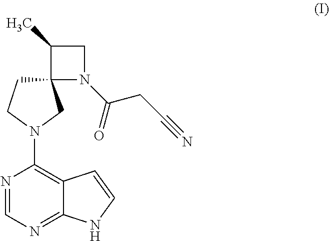 [SMILES: C[C@H]1CN(C(=O)CC#N)[C@]12CCN(c1ncnc3[nH]ccc13)C2]

3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof as an active ingredient.

Clause 28. The therapeutic or preventive agent according to any one of the preceding clauses 25-27 wherein the agent is a topical agent.

Clause 29. The therapeutic or preventive agent according to any one of the preceding clauses 25-28 wherein the agent is a cream.

Clause 30. The therapeutic or preventive agent according to any one of the preceding clauses 25-29 wherein the compound of formula (I) is administered as 20 mg/g concentration.

Clause 31. The therapeutic or preventive agent according to any one of the preceding clauses 25-30 wherein the compound of formula (I) is administered as a twice daily application.

Clause 32. The therapeutic or preventive agent according to any one of the preceding clauses 25-31 wherein the compound of formula (I) is administered for 12 weeks.

Clause 33. A method for treating cicatricial alopecia in a subject in need thereof, which method comprises the step of administering to said subject a therapeutically effective amount of the compound of formula (I).

Clause 34. A method for treating LPP in a subject in need thereof, which method comprises the step of administering to said subject a therapeutically effective amount of the compound of formula (I).

Clause 35. A method for treating FFA in a subject in need thereof, which method comprises the step of administering to said subject a therapeutically effective amount of the compound of formula (I).

Clause 36. The method according to any one of the preceding clauses 33-35 wherein the administration is topical.

Clause 37. The method according to any one of the preceding clauses 33-36 wherein the topical formulation is a cream.

Clause 38. The method according to any one of the preceding clauses 33-37 wherein the compound of formula (I) is administered in a concentration of 20 mg/g.

Clause 39. The method according to any one of the preceding clauses 33-38 wherein the compound of formula (I) is administered as a twice daily application.

Clause 40. The method according to any one of the preceding clause 33-39 wherein the compound of formula (I) is administered for 12 weeks.

Clause 41. A method of treating frontal fibrosing alopecia (FFA) in a human patient in need thereof comprising administration of a topical composition comprising delgocitinib on a free base basis or a pharmaceutically acceptable salt thereof.

Clause 42. The compound delgocitinib or a topical composition comprising delgocitinib for use in a method of treating frontal fibrosing alopecia (FFA) in a human patient in need thereof comprising administration of a topical composition comprising delgocitinib on a free base basis or a pharmaceutically acceptable salt thereof.

Clause 43. Use of the compound delgocitinib for the manufacture of a topical composition for treating frontal fibrosing alopecia (FFA) in a human patient in need thereof comprising administration of a topical composition comprising delgocitinib on a free base basis or a pharmaceutically acceptable salt thereof.

Clause 44. The method of treating, the compound or topical formulation for use or the use of the compound according to anyone of clauses 41-43, wherein the patient has a clinically confirmed diagnosis of FFA.

Clause 45. The method of treating, the compound or topical formulation for use or the use of the compound according to anyone of clauses 41-44, wherein the patient has a has a target area with a perifollicular erythema score 2 and a perifollicular scale score 2.

Clause 46. The method of treating, the compound or topical formulation for use or the use of the compound according to anyone of clauses 41-45, wherein the patient is 45 years or older.

Clause 47. The method of treating, the compound or topical formulation for use or the use of the compound according to anyone of clauses 41-46, wherein the patient is female.

Clause 48. The method of treating, the compound or topical formulation for use or the use of the compound according to anyone of clauses 41-47, wherein the patient is postmenopausal (as defined above)

Clause 49. The method of treating, the compound or topical formulation for use or the use of the compound according to anyone of clauses 41-48, wherein the patient does not have a history of other scalp/hair disease including discoid lupus erythematosus and central centrifugal cicatricial alopecia.

Clause 50. The method of treating, the compound or topical formulation for use or the use of the compound according to anyone of clauses 41-49, wherein the patient achieves a reduction in the expression of one or more of the biomarkers CXCL9, CXCL10 and/or IFN-γ from baseline at week 26.

Clause 51. The method of treating, the compound or topical formulation for use or the use of the compound according to anyone of clauses 41-49, wherein the patient achieves a reduction in the expression of one or more of the biomarkers CXCL9, CXCL10 and/or IFN-γ from baseline at week 24.

Clause 52. The method of treating, the compound or topical formulation for use or the use of the compound according to anyone of clauses 41-49, wherein the patient achieves a reduction in the expression of one or more of the biomarkers CXCL9, CXCL10 and/or IFN-γ from baseline at week 12.

Clause 53. The method of treating, the compound or topical formulation for use or the use of the compound according to anyone of clauses 41-49, wherein the patient achieves a reduction in the expression of one or more of the biomarkers CXCL9, CXCL10 and/or IFN-γ from baseline at week 8.

Clause 54. The method of treating, the compound or topical formulation for use or the use of the compound according to anyone of clauses 41-49, wherein the patient achieves a reduction in the expression of one or more of the biomarkers CXCL9, CXCL10 and/or IFN-γ from baseline at week 4.

Clause 55. The method of treating, the compound or topical formulation for use or the use of the compound according to anyone of clauses 41-54, wherein the patient achieves an improvement in LPPAI score from baseline at week 26.

Clause 56. The method of treating, the compound or topical formulation for use or the use of the compound according to anyone of clauses 41-54, wherein the patient achieves an improvement in LPPAI score from baseline at week 24.

Clause 57. The method of treating, the compound or topical formulation for use or the use of the compound according to anyone of clauses 41-54, wherein the patient achieves an improvement in LPPAI score from baseline at week 12.

Clause 58. The method of treating, the compound or topical formulation for use or the use of the compound according to anyone of clauses 41-54, wherein the patient achieves an improvement in LPPAI score from baseline at week 8.

Clause 59. The method of treating, the compound or topical formulation for use or the use of the compound according to anyone of clauses 41-54, wherein the patient achieves an improvement in LPPAI score from baseline at week 4.

Clause 60. The method of treating, the compound or topical formulation for use or the use of the compound according to anyone of clauses 41-59, wherein the patient achieves an improvement in FFASS score from baseline at week 26.

Clause 61. The method of treating, the compound or topical formulation for use or the use of the compound according to anyone of clauses 41-59, wherein the patient achieves an improvement in FFASS score from baseline at week 24.

Clause 62. The method of treating, the compound or topical formulation for use or the use of the compound according to anyone of clauses 41-59, wherein the patient achieves an improvement in FFASS score from baseline at week 12.

Clause 63. The method of treating, the compound or topical formulation for use or the use of the compound according to anyone of clauses 41-59, wherein the patient achieves an improvement in FFASS score from baseline at week 8.

Clause 64. The method of treating, the compound or topical formulation for use or the use of the compound according to anyone of clauses 41-59, wherein the patient achieves an improvement in FFASS score from baseline at week 4.

Clause 65. The method of treating, the compound or topical formulation for use or the use of the compound according to anyone of clauses 41-64, wherein the patient achieves an improvement in target area perifollicular erythema score from baseline at week 26.

Clause 66. The method of treating, the compound or topical formulation for use or the use of the compound according to anyone of clauses 41-64, wherein the patient achieves an improvement in target area perifollicular erythema score from baseline at week 24.

Clause 67. The method of treating, the compound or topical formulation for use or the use of the compound according to anyone of clauses 41-64, wherein the patient achieves an improvement in target area perifollicular erythema score from baseline at week 12.

Clause 68. The method of treating, the compound or topical formulation for use or the use of the compound according to anyone of clauses 41-64, wherein the patient achieves an improvement in target area perifollicular erythema score from baseline at week 8.

Clause 69. The method of treating, the compound or topical formulation for use or the use of the compound according to anyone of clauses 41-64, wherein the patient achieves an improvement in target area perifollicular erythema score from baseline at week 4.

Clause 70. The method of treating, the compound or topical formulation for use or the use of the compound according to anyone of clauses 41-69, wherein the patient achieves an improvement in target area perifollicular scale score from baseline at week 26.

Clause 71. The method of treating, the compound or topical formulation for use or the use of the compound according to anyone of clauses 41-69, wherein the patient achieves an improvement in target area perifollicular scale score from baseline at week 24.

Clause 72. The method of treating, the compound or topical formulation for use or the use of the compound according to anyone of clauses 41-69, wherein the patient achieves an improvement in target area perifollicular scale score from baseline at week 12.

Clause 73. The method of treating, the compound or topical formulation for use or the use of the compound according to anyone of clauses 41-69, wherein the patient achieves an improvement in target area perifollicular scale score from baseline at week 8.

Clause 74. The method of treating, the compound or topical formulation for use or the use of the compound according to anyone of clauses 41-69, wherein the patient achieves an improvement in target area perifollicular scale score from baseline at week 4.

Clause 75. The method of treating, the compound or topical formulation for use or the use of the compound according to anyone of clauses 41-74, wherein the patient achieves a reduction in pruritus NRS score from baseline at week 26.

Clause 76. The method of treating, the compound or topical formulation for use or the use of the compound according to anyone of clauses 41-74, wherein the patient achieves a reduction in pruritus NRS score from baseline at week 24.

Clause 77. The method of treating, the compound or topical formulation for use or the use of the compound according to anyone of clauses 41-74, wherein the patient achieves a reduction in pruritus NRS score from baseline at week 12.

Clause 78. The method of treating, the compound or topical formulation for use or the use of the compound according to anyone of clauses 41-74, wherein the patient achieves a reduction in pruritus NRS score from baseline at week 8.

Clause 79. The method of treating, the compound or topical formulation for use or the use of the compound according to anyone of clauses 41-74, wherein the patient achieves a reduction in pruritus NRS score from baseline at week 4.

Clause 80. The method of treating, the compound or topical formulation for use or the use of the compound according to anyone of clauses 41-74, wherein the patient achieves a reduction in pruritus NRS score from baseline at day 8.

Clause 81. The method of treating, the compound or topical formulation for use or the use of the compound according to anyone of clauses 41-74, wherein the patient achieves a reduction in pruritus NRS score from baseline at day 7.

Clause 82. The method of treating, the compound or topical formulation for use or the use of the compound according to anyone of clauses 41-74, wherein the patient achieves a reduction in pruritus NRS score from baseline at day 6.

Clause 83. The method of treating, the compound or topical formulation for use or the use of the compound according to anyone of clauses 41-74, wherein the patient achieves a reduction in pruritus NRS score from baseline at day 5.

Clause 84. The method of treating, the compound or topical formulation for use or the use of the compound according to anyone of clauses 41-74, wherein the patient achieves a reduction in pruritus NRS score from baseline at day 4.

Clause 85. The method of treating, the compound or topical formulation for use or the use of the compound according to anyone of clauses 41-74, wherein the patient achieves a reduction in pruritus NRS score from baseline at day 3.

Clause 86. The method of treating, the compound or topical formulation for use or the use of the compound according to anyone of clauses 41-74, wherein the patient achieves a reduction in pruritus NRS score from baseline at day 2.

Clause 87. The method of treating, the compound or topical formulation for use or the use of the compound according to anyone of clauses 41-86, wherein the patient achieves a reduction in burning sensation NRS score from baseline at week 26.

Clause 88. The method of treating, the compound or topical formulation for use or the use of the compound according to anyone of clauses 41-86, wherein the patient achieves a reduction in burning sensation NRS score from baseline at week 24.

Clause 89. The method of treating, the compound or topical formulation for use or the use of the compound according to anyone of clauses 41-86, wherein the patient achieves a reduction in burning sensation NRS score from baseline at week 12.

Clause 90. The method of treating, the compound or topical formulation for use or the use of the compound according to anyone of clauses 41-86, wherein the patient achieves a reduction in burning sensation NRS score from baseline at week 8.

Clause 91. The method of treating, the compound or topical formulation for use or the use of the compound according to anyone of clauses 41-86, wherein the patient achieves a reduction in burning sensation NRS score from baseline at week 4.

Clause 92. The method of treating, the compound or topical formulation for use or the use of the compound according to anyone of clauses 41-86, wherein the patient achieves a reduction in burning sensation NRS score from baseline at day 8.

Clause 93. The method of treating, the compound or topical formulation for use or the use of the compound according to anyone of clauses 41-86, wherein the patient achieves a reduction in burning sensation NRS score from baseline at day 7.

Clause 94. The method of treating, the compound or topical formulation for use or the use of the compound according to anyone of clauses 41-86, wherein the patient achieves a reduction in burning sensation NRS score from baseline at day 6.

Clause 95. The method of treating, the compound or topical formulation for use or the use of the compound according to anyone of clauses 41-86, wherein the patient achieves a reduction in burning sensation NRS score from baseline at day 5.

Clause 96. The method of treating, the compound or topical formulation for use or the use of the compound according to anyone of clauses 41-86, wherein the patient achieves a reduction in burning sensation NRS score from baseline at day 4.

Clause 97. The method of treating, the compound or topical formulation for use or the use of the compound according to anyone of clauses 41-86, wherein the patient achieves a reduction in burning sensation NRS score from baseline at day 3.

Clause 98. The method of treating, the compound or topical formulation for use or the use of the compound according to anyone of clauses 41-86, wherein the patient achieves a reduction in burning sensation NRS score from baseline at day 2.

Clause 99. The method of treating, the compound or topical formulation for use or the use of the compound according to anyone of clauses 41-98, wherein the patient achieves a reduction in pain NRS score from baseline at week 26.

Clause 100. The method of treating, the compound or topical formulation for use or the use of the compound according to anyone of clauses 41-98, wherein the patient achieves a reduction in pain NRS score from baseline at week 24.

Clause 101. The method of treating, the compound or topical formulation for use or the use of the compound according to anyone of clauses 41-98, wherein the patient achieves a reduction in pain NRS score from baseline at week 12.

Clause 102. The method of treating, the compound or topical formulation for use or the use of the compound according to anyone of clauses 41-98, wherein the patient achieves a reduction in pain NRS score from baseline at week 8.

Clause 103. The method of treating, the compound or topical formulation for use or the use of the compound according to anyone of clauses 41-98, wherein the patient achieves a reduction in pain NRS score from baseline at week 4.

Clause 104. The method of treating, the compound or topical formulation for use or the use of the compound according to anyone of clauses 41-98, wherein the patient achieves a reduction in pain NRS score from baseline at day 8.

Clause 105. The method of treating, the compound or topical formulation for use or the use of the compound according to anyone of clauses 41-98, wherein the patient achieves a reduction in pain NRS score from baseline at day 7.

Clause 106. The method of treating, the compound or topical formulation for use or the use of the compound according to anyone of clauses 41-98, wherein the patient achieves a reduction in pain NRS score from baseline at day 6.

Clause 107. The method of treating, the compound or topical formulation for use or the use of the compound according to anyone of clauses 41-98, wherein the patient achieves a reduction in pain NRS score from baseline at day 5.

Clause 108. The method of treating, the compound or topical formulation for use or the use of the compound according to anyone of clauses 41-98, wherein the patient achieves a reduction in pain NRS score from baseline at day 4.

Clause 109. The method of treating, the compound or topical formulation for use or the use of the compound according to anyone of clauses 41-98, wherein the patient achieves a reduction in pain NRS score from baseline at day 3.

Clause 110. The method of treating, the compound or topical formulation for use or the use of the compound according to anyone of clauses 41-98, wherein the patient achieves a reduction in pain NRS score from baseline at day 2.

Clause 111. The method of treating, the compound or topical formulation for use or the use of the compound according to anyone of clauses 41-110, wherein the patient has a baseline pruritus NRS score 3 and achieves a 3-point reduction in pruritus NRS score at week 26.

Clause 112. The method of treating, the compound or topical formulation for use or the use of the compound according to anyone of clauses 41-110, wherein the patient has a baseline pruritus NRS score 3 and achieves a 3-point reduction in pruritus NRS score at week 24.

Clause 113. The method of treating, the compound or topical formulation for use or the use of the compound according to anyone of clauses 41-110, wherein the patient has a baseline pruritus NRS score 3 and achieves a 3-point reduction in pruritus NRS score at week 12.

Clause 114. The method of treating, the compound or topical formulation for use or the use of the compound according to anyone of clauses 41-110 wherein the patient has a baseline pruritus NRS score 3 and achieves a 3-point reduction in pruritus NRS score at week 8.

Clause 115. The method of treating, the compound or topical formulation for use or the use of the compound according to anyone of clauses 41-110 wherein the patient has a baseline pruritus NRS score 3 and achieves a 3-point reduction in pruritus NRS score at week 4.

Clause 116. The method of treating, the compound or topical formulation for use or the use of the compound according to anyone of clauses 41-110 wherein the patient has a baseline pruritus NRS score 3 and achieves a 3-point reduction in pruritus NRS score at day 8.

Clause 117. The method of treating, the compound or topical formulation for use or the use of the compound according to anyone of clauses 41-110 wherein the patient has a baseline pruritus NRS score 3 and achieves a 3-point reduction in pruritus NRS score at day 7.

Clause 118. The method of treating, the compound or topical formulation for use or the use of the compound according to anyone of clauses 41-110 wherein the patient has a baseline pruritus NRS score 3 and achieves a 3-point reduction in pruritus NRS score at day 6.

Clause 119. The method of treating, the compound or topical formulation for use or the use of the compound according to anyone of clauses 41-110 wherein the patient has a baseline pruritus NRS score 3 and achieves a 3-point reduction in pruritus NRS score at day 5.

Clause 120. The method of treating, the compound or topical formulation for use or the use of the compound according to anyone of clauses 41-110 wherein the patient has a baseline pruritus NRS score 3 and achieves a 3-point reduction in pruritus NRS score at day 4.

Clause 121. The method of treating, the compound or topical formulation for use or the use of the compound according to anyone of clauses 41-110 wherein the patient has a baseline pruritus NRS score 3 and achieves a 3-point reduction in pruritus NRS score at day 3.

Clause 122. The method of treating, the compound or topical formulation for use or the use of the compound according to anyone of clauses 41-110 wherein the patient has a baseline pruritus NRS score 3 and achieves a 3-point reduction in pruritus NRS score at day 2.

Clause 123. The method of treating, the compound or topical formulation for use or the use of the compound according to anyone of clauses 41-122, wherein the patient has a baseline pruritus NRS score 4 and achieves a 4 point reduction in pruritus NRS score at week 26.

Clause 124. The method of treating, the compound or topical formulation for use or the use of the compound according to anyone of clauses 41-122, wherein the patient has a baseline pruritus NRS score 4 and achieves a 4-point reduction in pruritus NRS score at week 24.

Clause 125. The method of treating, the compound or topical formulation for use or the use of the compound according to anyone of clauses 41-122, wherein the patient has a baseline pruritus NRS score 4 and achieves a 4-point reduction in pruritus NRS score at week 12.

Clause 126. The method of treating, the compound or topical formulation for use or the use of the compound according to anyone of clauses 41-122 wherein the patient has a baseline pruritus NRS score 4 and achieves a 4-point reduction in pruritus NRS score at week 8.

Clause 127. The method of treating, the compound or topical formulation for use or the use of the compound according to anyone of clauses 41-122 wherein the patient has a baseline pruritus NRS score 4 and achieves a 4-point reduction in pruritus NRS score at week 4.

Clause 128. The method of treating, the compound or topical formulation for use or the use of the compound according to anyone of clauses 41-122 wherein the patient has a baseline pruritus NRS score 4 and achieves a 4-point reduction in pruritus NRS score at day 8.

Clause 129. The method of treating, the compound or topical formulation for use or the use of the compound according to anyone of clauses 41-122 wherein the patient has a baseline pruritus NRS score 4 and achieves a 4-point reduction in pruritus NRS score at day 7.

Clause 130. The method of treating, the compound or topical formulation for use or the use of the compound according to anyone of clauses 41-122 wherein the patient has a baseline pruritus NRS score 4 and achieves a 4-point reduction in pruritus NRS score at day 6.

Clause 131. The method of treating, the compound or topical formulation for use or the use of the compound according to anyone of clauses 41-122 wherein the patient has a baseline pruritus NRS score 4 and achieves a 4-point reduction in pruritus NRS score at day 5.

Clause 132. The method of treating, the compound or topical formulation for use or the use of the compound according to anyone of clauses 41-122 wherein the patient has a baseline pruritus NRS score 4 and achieves a 4-point reduction in pruritus NRS score at day 4.

Clause 133. The method of treating, the compound or topical formulation for use or the use of the compound according to anyone of clauses 41-122 wherein the patient has a baseline pruritus NRS score 4 and achieves a 4-point reduction in pruritus NRS score at day 4.

Clause 134. The method of treating, the compound or topical formulation for use or the use of the compound according to anyone of clauses 41-122 wherein the patient has a baseline pruritus NRS score 4 and achieves a 4-point reduction in pruritus NRS score at day 2.

Clause 135. The method of treating, the compound or topical formulation for use or the use of the compound according to anyone of clauses 41-134, wherein the patient has a baseline burning sensation NRS score 3 and achieves a 3-point reduction in burning sensation NRS score at week 26.

Clause 136. The method of treating, the compound or topical formulation for use or the use of the compound according to anyone of clauses 41-134, wherein the patient has a baseline burning sensation NRS score 3 and achieves a 3-point reduction in burning sensation NRS score at week 24.

Clause 137. The method of treating, the compound or topical formulation for use or the use of the compound according to anyone of clauses 41-134, wherein the patient has a baseline burning sensation NRS score 3 and achieves a 3-point reduction in burning sensation NRS score at week 12.

Clause 138. The method of treating, the compound or topical formulation for use or the use of the compound according to anyone of clauses 41-134 wherein the patient has a baseline burning sensation NRS score 3 and achieves a 3-point reduction in burning sensation NRS score at week 8.

Clause 139. The method of treating, the compound or topical formulation for use or the use of the compound according to anyone of clauses 41-134 wherein the patient has a baseline burning sensation NRS score 3 and achieves a 3-point reduction in burning sensation NRS score at week 4.

Clause 140. The method of treating, the compound or topical formulation for use or the use of the compound according to anyone of clauses 41-134 wherein the patient has a baseline burning sensation NRS score 3 and achieves a 3-point reduction in burning sensation NRS score at day 8.

Clause 141. The method of treating, the compound or topical formulation for use or the use of the compound according to anyone of clauses 41-134 wherein the patient has a baseline burning sensation NRS score 3 and achieves a 3-point reduction in burning sensation NRS score at day 7.

Clause 142. The method of treating, the compound or topical formulation for use or the use of the compound according to anyone of clauses 41-134 wherein the patient has a baseline burning sensation NRS score 3 and achieves a 3-point reduction in burning sensation NRS score at day 6.

Clause 143. The method of treating, the compound or topical formulation for use or the use of the compound according to anyone of clauses 41-134 wherein the patient has a baseline burning sensation NRS score 3 and achieves a 3-point reduction in burning sensation NRS score at day 5.

Clause 144. The method of treating, the compound or topical formulation for use or the use of the compound according to anyone of clauses 41-134 wherein the patient has a baseline burning sensation NRS score 3 and achieves a 3-point reduction in burning sensation NRS score at day 4.

Clause 145. The method of treating, the compound or topical formulation for use or the use of the compound according to anyone of clauses 41-134 wherein the patient has a baseline burning sensation NRS score 3 and achieves a 3-point reduction in burning sensation NRS score at day 3.

Clause 146. The method of treating, the compound or topical formulation for use or the use of the compound according to anyone of clauses 41-134 wherein the patient has a baseline burning sensation NRS score 3 and achieves a 3-point reduction in burning sensation NRS score at day 2.

Clause 147. The method of treating, the compound or topical formulation for use or the use of the compound according to anyone of clauses 41-146, wherein the patient has a baseline burning sensation NRS score 4 and achieves a 4-point reduction in burning sensation NRS score at week 26.

Clause 148. The method of treating, the compound or topical formulation for use or the use of the compound according to anyone of clauses 41-146, wherein the patient has a baseline burning sensation NRS score 4 and achieves a 4-point reduction in burning sensation NRS score at week 24.

Clause 149. The method of treating, the compound or topical formulation for use or the use of the compound according to anyone of clauses 41-146, wherein the patient has a baseline burning sensation NRS score 4 and achieves a 4-point reduction in burning sensation NRS score at week 12.

Clause 150. The method of treating, the compound or topical formulation for use or the use of the compound according to anyone of clauses 41-146 wherein the patient has a baseline burning sensation NRS score 4 and achieves a 4-point reduction in burning sensation NRS score at week 8.

Clause 151. The method of treating, the compound or topical formulation for use or the use of the compound according to anyone of clauses 41-146 wherein the patient has a baseline burning sensation NRS score 4 and achieves a 4-point reduction in burning sensation NRS score at week 4.

Clause 152. The method of treating, the compound or topical formulation for use or the use of the compound according to anyone of clauses 41-146 wherein the patient has a baseline burning sensation NRS score 4 and achieves a 4-point reduction in burning sensation NRS score at day 8.

Clause 153. The method of treating, the compound or topical formulation for use or the use of the compound according to anyone of clauses 41-146 wherein the patient has a baseline burning sensation NRS score 4 and achieves a 4-point reduction in burning sensation NRS score at day 7.

Clause 154. The method of treating, the compound or topical formulation for use or the use of the compound according to anyone of clauses 41-146 wherein the patient has a baseline burning sensation NRS score 4 and achieves a 4-point reduction in burning sensation NRS score at day 6.

Clause 155. The method of treating, the compound or topical formulation for use or the use of the compound according to anyone of clauses 41-146 wherein the patient has a baseline burning sensation NRS score 4 and achieves a 4-point reduction in burning sensation NRS score at day 5.

Clause 156. The method of treating, the compound or topical formulation for use or the use of the compound according to anyone of clauses 41-146 wherein the patient has a baseline burning sensation NRS score 4 and achieves a 4-point reduction in burning sensation NRS score at day 4.

Clause 157. The method of treating, the compound or topical formulation for use or the use of the compound according to anyone of clauses 41-146 wherein the patient has a baseline burning sensation NRS score 4 and achieves a 4-point reduction in burning sensation NRS score at day 3.

Clause 158. The method of treating, the compound or topical formulation for use or the use of the compound according to anyone of clauses 41-134 wherein the patient has a baseline burning sensation NRS score 4 and achieves a 4-point reduction in burning sensation NRS score at day 2.

Clause 159. The method of treating, the compound or topical formulation for use or the use of the compound according to anyone of clauses 41-158, wherein the patient has a baseline pain NRS score 3 and achieves a 3-point reduction in pain NRS score at week 26.

Clause 160. The method of treating, the compound or topical formulation for use or the use of the compound according to anyone of clauses 41-158, wherein the patient has a baseline pain NRS score 3 and achieves a 3-point reduction in pain NRS score at week 24.

Clause 161. The method of treating, the compound or topical formulation for use or the use of the compound according to anyone of clauses 41-158, wherein the patient has a baseline pain NRS score 3 and achieves a 3-point reduction in pain NRS score at week 12.

Clause 162. The method of treating, the compound or topical formulation for use or the use of the compound according to anyone of clauses 41-158 wherein the patient has a baseline pain NRS score 3 and achieves a 3-point reduction in pain NRS score at week 8.

Clause 163. The method of treating, the compound or topical formulation for use or the use of the compound according to anyone of clauses 41-158 wherein the patient has a baseline pain NRS score 3 and achieves a 3-point reduction in pain NRS score at week 4.

Clause 164. The method of treating, the compound or topical formulation for use or the use of the compound according to anyone of clauses 41-158 wherein the patient has a baseline pain NRS score 3 and achieves a 3-point reduction in pain NRS score at day 8.

Clause 165. The method of treating, the compound or topical formulation for use or the use of the compound according to anyone of clauses 41-158 wherein the patient has a baseline pain NRS score 3 and achieves a 3-point reduction in pain NRS score at day 7.

Clause 166. The method of treating, the compound or topical formulation for use or the use of the compound according to anyone of clauses 41-158 wherein the patient has a baseline pain NRS score 3 and achieves a 3-point reduction in pain NRS score at day 6.

Clause 167. The method of treating, the compound or topical formulation for use or the use of the compound according to anyone of clauses 41-158 wherein the patient has a baseline pain NRS score 3 and achieves a 3-point reduction in pain NRS score at day 5.

Clause 168. The method of treating, the compound or topical formulation for use or the use of the compound according to anyone of clauses 41-158 wherein the patient has a baseline pain NRS score 3 and achieves a 3-point reduction in pain NRS score at day 4.

Clause 169. The method of treating, the compound or topical formulation for use or the use of the compound according to anyone of clauses 41-158 wherein the patient has a baseline pain NRS score 3 and achieves a 3-point reduction in pain NRS score at day 3.

Clause 170. The method of treating, the compound or topical formulation for use or the use of the compound according to anyone of clauses 41-158 wherein the patient has a baseline pain NRS score 3 and achieves a 3-point reduction in pain NRS score at day 2.

Clause 171. The method of treating, the compound or topical formulation for use or the use of the compound according to anyone of clauses 41-170, wherein the patient has a baseline pain NRS score 4 and achieves a 4-point reduction in pain NRS score at week 26.

Clause 172. The method of treating, the compound or topical formulation for use or the use of the compound according to anyone of clauses 41-170, wherein the patient has a baseline pain NRS score 4 and achieves a 4-point reduction in pain NRS score at week 24.

Clause 173. The method of treating, the compound or topical formulation for use or the use of the compound according to anyone of clauses 41-170, wherein the patient has a baseline pain NRS score 4 and achieves a 4-point reduction in pain NRS score at week 12.

Clause 174. The method of treating, the compound or topical formulation for use or the use of the compound according to anyone of clauses 41-170 wherein the patient has a baseline pain NRS score 4 and achieves a 4-point reduction in pain NRS score at week 8.

Clause 175. The method of treating, the compound or topical formulation for use or the use of the compound according to anyone of clauses 41-170 wherein the patient has a baseline pain NRS score 4 and achieves a 4-point reduction in pain NRS score at week 4.

Clause 176. The method of treating, the compound or topical formulation for use or the use of the compound according to anyone of clauses 41-170 wherein the patient has a baseline pain NRS score 4 and achieves a 4-point reduction in pain NRS score at day 8.

Clause 177. The method of treating, the compound or topical formulation for use or the use of the compound according to anyone of clauses 41-170 wherein the patient has a baseline pain NRS score 4 and achieves a 4-point reduction in pain NRS score at day 7.

Clause 178. The method of treating, the compound or topical formulation for use or the use of the compound according to anyone of clauses 41-170 wherein the patient has a baseline pain NRS score 4 and achieves a 4-point reduction in pain NRS score at day 6.

Clause 179. The method of treating, the compound or topical formulation for use or the use of the compound according to anyone of clauses 41-170 wherein the patient has a baseline pain NRS score 4 and achieves a 4-point reduction in pain NRS score at day 5.

Clause 180. The method of treating, the compound or topical formulation for use or the use of the compound according to anyone of clauses 41-170 wherein the patient has a baseline pain NRS score 4 and achieves a 4-point reduction in pain NRS score at day 4.

Clause 181. The method of treating, the compound or topical formulation for use or the use of the compound according to anyone of clauses 41-170 wherein the patient has a baseline pain NRS score 4 and achieves a 4-point reduction in pain NRS score at day 3.

Clause 182. The method of treating, the compound or topical formulation for use or the use of the compound according to anyone of clauses 41-170 wherein the patient has a baseline pain NRS score 4 and achieves a 4-point reduction in pain NRS score at day 2.

Clause 182. The method of treating, the compound or topical formulation for use or the use of the compound according to anyone of clauses 41-170, wherein the patient has a baseline pain NRS score 4 and achieves a 4-point reduction in pain NRS score at day 2.

Clause 183. The method of treating, the compound or topical formulation for use or the use of the compound according to anyone of clauses 41-182, wherein the patient achieves an improvement in target area hair counts/trichoscopy via fotofinder trichovision from baseline to week 26.

Clause 184. The method of treating, the compound or topical formulation for use or the use of the compound according to anyone of clauses 41-182, wherein the patient achieves an improvement in target area hair counts/trichoscopy via fotofinder trichovision from baseline to week 24.

Clause 185. The method of treating, the compound or topical formulation for use or the use of the compound according to anyone of clauses 41-182, wherein the patient achieves an improvement in target area hair counts/trichoscopy via fotofinder trichovision from baseline to week 12.

Clause 186. The method of treating, the compound or topical formulation for use or the use of the compound according to anyone of clauses 41-185, wherein the administration of the compound or topical formulation is twice daily.

Clause 187. The method of treating, the compound or topical formulation for use or the use of the compound according to anyone of clauses 41-185, wherein the administration of the topical formulation is twice daily and the topical composition comprises 20 mg/g delgocitnib on a free base basis or a pharmaceutically acceptable salt thereof.

Clause 188. The method of treating, the compound or topical formulation for use or the use of the compound according to anyone of clauses 41-187, wherein the topical composition is a water-in-oil emulsion.

Clause 189. The method of treating, the compound or topical formulation for use or the use of the compound according to anyone of clauses 41-187, wherein the topical composition is an acidified water-in-oil emulsion.

Clause 190. The method of treating, the compound or topical formulation for use or the use of the compound according to anyone of clauses 41-187, wherein the pH of the topical composition is between is about 3.8 and 4.6.

Clause 191. The method of treating, the compound or topical formulation for use or the use of the compound according to anyone of clauses 41-187, wherein the pH of the topical composition is between is about 4.4 or below.

Clause 192. The method of treating, the compound or topical formulation for use or the use of the compound according to anyone of clauses 41-187, wherein the pH of the topical composition is between is about 4.3 or below.

Clause 193. The method of treating, the compound or topical formulation for use or the use of the compound according to anyone of clauses 41-187, wherein the pH of the topical composition is between is about 4.2 or below.

Clause 194. The method of treating, the compound or topical formulation for use or the use of the compound according to anyone of clauses 188-193, wherein the topical composition comprises an oily base.

Clause 195. The method of treating, the compound or topical formulation for use or the use of the compound according to anyone of clauses 188-194, wherein the topical composition comprises an oily base and the oily base is liquid paraffin.

Clause 196. The method of treating, the compound or topical formulation for use or the use of the compound according to anyone of clauses 41-195, wherein the method of treating is preventive treatment, the compound or topical formulation for use is for preventive use or the use of the compound is for preventive use.

1. A compound of general formula (I) 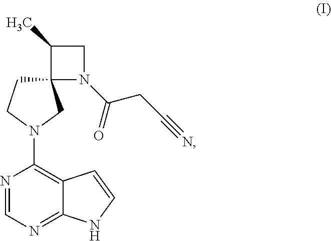
3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof for use in the treatment of LPP.

2. The compound for use according to claim 1 wherein the treatment is for FFA.

3. The compound for use according to any one of the preceding claims wherein the compound of formula (I) or a pharmaceutically acceptable salt thereof is administered as a cream.

4. The compound for use according to any one of the preceding claims wherein the compound of formula (I), or a pharmaceutically acceptable salt thereof is administered in a concentration of 20 mg/g.

5. A pharmaceutical composition for use in the treatment of LPP comprising a compound of formula (I) 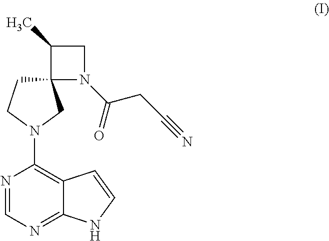 [SMILES: C[C@H]1CN(C(=O)CC#N)[C@]12CCN(c1ncnc3[nH]ccc13)C2]
3-[(3S,4R)-3-methyl-6-(7H-pyrrolo[2,3d]pyrimidin-4-yl)-1,6-diazaspiro[3,4]octan-1-yl]-3-oxopropanenitrile, or a pharmaceutically acceptable salt thereof, as an active ingredient.

6. The pharmaceutical composition for use according to claim 5, wherein the treatment is FFA.

7. The pharmaceutical composition for use in the treatment according to any one of the preceding claims 5-6 wherein the pharmaceutical composition is a cream.

8. The pharmaceutical composition for use in the treatment according to any one of the preceding claims 5-7 wherein the compound of formula (I), or a pharmaceutically acceptable salt thereof is administered in a concentration of 20 mg/g.
